(19) United States
(12) Patent Application Publication
(10) Pub. No.: US 2012/0270257 A1
(43) Pub. Date: Oct. 25, 2012

(54) Observation Cell Arrangement

(76) Inventors: Bryan Morris, Nottingham (GB); Tim Self, Nottingham (GB); Stephen John Hill, Nottingham (GB)

(21) Appl. No.: 13/500,262

(22) PCT Filed: Oct. 8, 2010
(86) PCT No.: PCT/GB2010/051698

§ 371 Date: Jun. 27, 2012

(30) Foreign Application Priority Data
Oct. 8, 2009 (GB) 0917565.4

(51) Int. Cl. C12M 1/34; G01N 21/64; G01N 21/00

(52) U.S. Cl. 435/29; 435/288.7

(57) ABSTRACT

An observation cell arrangement for flow perfusion of a sample to be examined, the arrangement comprising a flow cell (21) having a cavity therein to receive the sample, the flow cell (21) arranged to receive a flow of fluid through the cavity that is directed over the sample from a cavity inlet (22) to a cavity outlet (23), the cavity inlet (22) associated with a fluid supply line, and a first flow supply path (24) connected to the fluid supply line via a valve (39), the first flow supply path (24) adapted to receive pressure from a pressure source comprising a pressure reservoir (29) to drive fluid flow through the cavity at a desired flow rate

The present invention relates to an observation cell arrangement. In particular, it relates to an observation cell arrangement in which nutrient fluid flows are perfused to maintain cultured cells under observation. Further, it relates to a method of performing observations with flow perfusion and to a method of identifying drugs.

Perfusion systems are used for a range of live cell applications requiring a continuous flow of nutrient media.

Confocal microscopy is a technique utilised to increase micrograph contrast and/or to reconstruct three dimensional images by effectively eliminating out of focus light in specimens which are thicker than the notional focal plane. Such techniques are popular in life sciences where changes in cells require observation. It will be understood in conventional microscopy, that is to say wide field fluorescent microscopy, that an entire specimen is flooded with light from a light source. All parts of the specimen in the optical plane in such circumstances are excited and the resulting fluorescence detected by the photo detector or camera. In a confocal microscope there is point illumination and an effective pinhole is created in an optically conjugate plane in front of the detector to eliminate out of focus information. In such circumstances only light produced by fluorescence very close to the focal plane can be detected and consequently images are achieved that are better than for wide field microscopes. However, by using such a technique, much of the light from the sample fluorescence is blocked. Thus, in order to achieve adequate signal intensity longer exposures are typically required. To obtain good images and measurements while using longer exposures requires the sample under observation to be subjected to very stable conditions.

When cultured cells are examined they should be subject to consistent environmental conditions for best results. Provision of a continuous but uneven flow of fresh media to support the cultured cells may itself create changes in the image of cells as viewed through a confocal microscope or simply obliterate the image created. It will be understood that it is not only important to maintain a steady flow of media to support the cultured cells but a steady temperature, pH and composition such as with regard to oxygen levels etc. for consistency as a baseline for observations. A number of processes for delivery of media to support cultured cells are known including utilisation of peristaltic pumps. Peristaltic pumps unfortunately create pressure pulses in the delivered flow and therefore deviate from the desirable consistent laminar flow of media. Earlier techniques with regard to conventional observation cell arrangements for microscopes also have inherent problems. For example utilisation of a simple gravity fed pressure system means it is difficult to maintain the medium at a desired temperature, the pressure exerted may be dependent upon the volume of media in the reservoir and it is difficult to connect such a system to a pressure pump. Other techniques utilise syringe pump systems and again there are difficulties with regard to relying on one pump to control all the separate reservoirs, that is to say all of the syringe cylinders and maintaining the same temperature in each reservoir defined by the syringe cylinders. The use of syringe pumps is also expensive.

In view of the above it will be appreciated that it is difficult to provide a consistent laminar flow of media for cultured cells or similar subjects of observation. Additionally it will be understood that normally it will be desirable to see the reaction of cultured cells to external changes such as exposure to discrete quantities of drugs in the media flowing towards the cultured cells without again causing perturbations in the image due to switching between the base or systemic flow and the dosing flow of a drug or other change from the systemic flow.

According to a first aspect of the invention we provide an observation cell arrangement for flow perfusion of a sample to be observed, the arrangement comprising a flow cell having a cavity therein to receive the sample, the cavity having a cavity inlet and a cavity outlet, the flow cell arranged to receive a flow of fluid through the cavity from the inlet to the outlet that is directed over the sample, the cavity inlet associated with a fluid delivery line, and a first flow supply path connected to the fluid delivery line via a valve, the arrangement including a pressure source to pressurise the first flow supply path, the pressure source comprising a reservoir.

This is advantageous as the reservoir acts as a buffer, storing a volume of pressurised fluid to absorb pressure pulses from a pump, for example, which would affect the fluid flow through the flow cell. Further the reservoir helps maintain the apparatus at a steady temperature as the temperature of the air, or any other fluid that is pumped into the reservoir has time to equalise with the air/fluid already present in the reservoir.

Preferably, the reservoir receives pressure from a pump, the reservoir adapted to substantially reduce pressure pulses from the pump. Thus, the size of the reservoir can be selected depending on the flow rate that is required and also depending on the pump that is used.

As the reservoir is able to absorb pressure pulses, it does not have to be of a size sufficient to complete a full test before being recharged. Thus, liquid can be flowed through the flow cell over several days, which may be necessary in certain tests, and the reservoir can be recharged during this period without substantially affecting the flow through the flow cell.

Preferably, the first supply path comprises a first fluid vessel, the fluid vessel including a diaphragm adapted to drive fluid flow when acted on by pressure received from the reservoir. The diaphragm forms a “gas pressurised displacement member” that is particularly advantageous as it provides a cost effective way of transferring pressure to the fluid of the first vessel. Further, the diaphragm ensures that the driving fluid i.e. pressurised air does not contaminate the fluid, such as systemic fluid, that is present in the first vessel as it provides an impermeable barrier.

Preferably, the first supply path comprises a systemic supply path and the first fluid vessel is adapted to receive a systemic fluid. This is advantageous as the apparatus can maintain cultured cells present in the flow cell and allow them to be observed with improved reliability.

Preferably, the arrangement includes at least one further supply path connected to the fluid delivery line via a valve, the or each further supply path connected to a further pressure source. This is advantageous as the further supply path can selectively deliver different fluid to the flow cell. The first pressure source and further pressure source may comprise the same pressure source. Preferably, the or each further supply path is adapted to supply the pressure to act directly on the contents of the further supply path. Alternatively, the or each further supply path may comprise a fluid vessel and a diaphragm adapted to drive fluid flow from the fluid vessel when acted on by pressure received from the pressure source.

Preferably, the or each further supply path includes a well between its associated valve and the fluid delivery line, the well arranged to reduce pressure pulses on actuation of the valve. This is advantageous, as the well is able to receive a flow of fluid before it enters the fluid delivery line which assists in ensuring a smooth flow rate when the further supply path is opened. Preferably the or each further supply path are arranged to connect to the fluid delivery line at an angle greater than 90° and less than 180°. Preferably the angle of convergence between the further supply path and the fluid delivery line is substantially 120°. This has been found to assist in providing smooth flow.

Preferably, the at least one further supply path comprises a dosing supply path for receiving a drug to be introduced into the cavity. This is advantageous as the apparatus can be used to observe the effect of drugs on cultured cells and for the identification of effective drugs.

Preferably, the reservoir comprises a reservoir of pressurized air. Using pressurized air results in an apparatus that requires the minimum of external connections and supplies. The diaphragm ensures that the air does not contaminate the systemic fluid, for example.

Preferably, the flow cell includes an observation window adapted to receive an examination device comprising a confocal microscope for examining the sample contained in the cavity or a fluorescence detector arranged to detect light emitted by the sample contained in the cavity or a other suitable detector. This is advantageous as the smooth fluid flow that the apparatus provides ensures reliable observations and/or measurements can be made by either the microscope or other detectors. The output from the detectors may be an image, a series of images, measurements, a graph or any other appropriate output or combination of outputs. It will be appreciated that any appropriate type of detector can be used to collect data through the observation window, as it is the apparatus that allows the presentation of a sample which is well sustained, but not disturbed by fluid flow.

Preferably, the or each of the supply paths includes a flow regulator. This is advantageous as the flow regulator ensures that a substantially constant pressure is supplied to the fluid supply paths.

Preferably, the further supply paths are supplied with pressure via a common manifold. This provides a simple connection for further supply paths to be added to the arrangement.

Preferably, the flow cell comprises a ring between two cover plate elements. Preferably, the cavity inlet has a plurality of injectors spaced around the periphery of the cavity. The injectors may be directed towards a centre of the cavity or are parallel to each other. The injectors may be of different sizes.

Preferably, the inlet is configured to provide a substantively laminar fluid flow across the flow cell from one side to the other. Preferably, the cavity is round or diamond shaped or oval.

Preferably, the arrangement is housed in an environmental cabinet to maintain the arrangement at a substantially constant temperature. This is advantageous as temperature gradients can have a detrimental effect on reliability. As the apparatus uses pressurized air and a preloaded vessels of fluid, only an electricity connection is required, which makes mounting the arrangement in a temperature controlled box easier.

A method of performing observations with a flow perfusion apparatus comprising the steps of;
a) adding a systemic fluid to a first fluid vessel;
b) charging a reservoir with pressurised fluid;
c) adding a sample to be observed to a flow cell cavity;
d) initiating a flow of fluid through flow cell the cavity using the pressurised fluid from the reservoir;
e) examining the sample.

This is advantageous as the observations (which can include measurements, counting, imaging, and viewing) are performed in a reliable consistent environment with smooth flow perfusion of the systemic fluid through the flow cell. Further, as the pressurised fluid is typically air, the method is easy to perform due to the minimum of external connections.

Preferably step (e) comprises using a confocal microscope to observe the sample or using a florescence detector to detect fluorescence emitted by the sample.

The method may include the step of making adjustments to the flow rate of at least the fluid in the first fluid vessel and making further examinations.

The apparatus may include a further fluid supply path containing a dosing fluid, and the method comprising the steps of;
i) introducing the dosing fluid and
ii) examining the effect of the dosing fluid on the sample.

Preferably the method includes the step of temporarily reducing the flow rate of the systemic fluid prior to the introduction of the dosing fluid and upon introduction of the dosing fluid, increasing the flow rate to provide a cue to an observer that the dosing fluid has been introduced. Preferably, the method includes increasing the flow rate to a level substantially equal to that prior to the temporary reduction in flow rate.

Preferably, the method includes the step of introducing dosing fluid in addition to the systemic fluid, while maintaining a substantially constant flow rate through the flow cell when the dosing fluid is introduced.

Preferably, the method includes the step of simultaneously actuating the valves associated with the systemic fluid and the dosing fluid, when the dosing fluid is introduced, thus maintaining a substantially constant flow rate through the flow cell when the dosing fluid is introduced.

Preferably, the method includes the step of connecting the apparatus only to one external supply, namely an electricity supply, prior to observing the sample.

Preferably the method includes the step of charging the reservoir with pressurised fluid while the fluid is flowing through the flow cell. This step is possible as the reservoir can absorb any pressure pulses caused by a pump or the like that charges it with pressurised fluid.

Preferably the method is performed using the apparatus of the first aspect of the invention.

According to a third aspect of the invention, we provide a method of identifying or studying drugs comprising the steps of;
a) placing cells in a flow cell;
b) providing a systemic fluid flow at a first, substantially constant, flow rate over the cells;
c) introducing a dosing fluid flow of a first drug while maintaining the substantially constant first flow rate over the cells;
d) observing the effect of the first drug on the cells;
e) identifying the drug if the effect on the cells fulfils predetermined criteria.

This is advantageous as the method provides a reliable way of identifying drugs as the drug's interaction with the cells can be easily monitored.

The method may include performing a kinetic test. In particular, the cells may be subject to chemicals or antibodies or proteins and how the chemicals bind and unbind to receptors may be measured/observed.

Alternatively, the method may include introducing a first substance to perfuse the cells, and observing the effect on the cells, introducing a second substance to perfuse the cells in addition to the first substance and observing how the second substance influences the effect of the first substance on the cells.

Preferably the method includes the step of introducing a first substance to perfuse the cells, diluting the first substance during a “washout” phase, and observing the effect on the cells. Preferably this method step additionally includes introducing a second substance, different to the first substance, during the “washout” phase and observing the resulting allosteric effect on the cells. Thus, a specific application of this allows for conditions of infinite dilution to be applied so that the “washout” of the first substance from the cells can be monitored in real time. In addition, if a second substance is applied during this “washout” phase, the allosteric effect of a substance (acting at a separate site on a cell membrane protein to the first substance i.e. acting at an allosteric site) on the washout of the first substance can be monitored. This test is particularly advantageous as the substances can be introduced and withdrawn over time so the changes can be observed in real time. The apparatus of the first aspect ensures that the introduction and removal of substances can be done smoothly and reliably.

Preferably the flow rate of the systemic fluid is temporarily reduced prior to the introduction of the dosing fluid and upon introduction of the dosing fluid, the flow rate is increased to provide a cue to an observer that the dosing fluid has been introduced.

Preferably the systemic fluid flow is provided by a reservoir of pressurised air. Preferably the method includes the step of charging the reservoir with pressurised fluid while the fluid is flowing through the flow cell. This step is possible as the reservoir can absorb any pressure pulses caused by a pump or the like that charges it with pressurised fluid.

It will be appreciated that the optional features of the second aspect of the invention apply equally to the third aspect of the invention.

Also in accordance with aspects of the present invention there is provided an observation cell arrangement for a microscope, the arrangement comprising a flow cell having a cavity between an inlet and an outlet, a vessel for fluid coupled to the flow cell, the vessel having a diaphragm to pressurise fluids therein and a size relative to the cavity whereby a flow rate between the inlet and the outlet is substantially maintained at least in an observation portion of the flow cell for a period of time.

In accordance with aspects of the present invention there is provided an observation cell arrangement for a microscope, the arrangement comprising a flow cell having a cavity to receive a fluid flow, the cavity having an inlet and an outlet, the inlet associated with a fluid supply comprising a systemic supply path and a dosing supply path, each supply path associated with the inlet by a valve and having a common pressurisation source to drive fluid flow to fill the cavity at a desired flow rate through a parallel coupling to the inlet and then out of the outlet, the systemic supply path and the dosing supply path configured to be substantially balanced in terms of flow presented to the cavity whereby closure of the valve in the systemic supply path and simultaneous opening of the valve in the dosing supply path substantially maintains the desired flow rate in the cavity.

Typically, there is a plurality of dosing supply paths. Generally, the common pressurisation source is an air pressure reservoir. Typically, the systemic supply path includes a fluid vessel and a supply diaphragm. Possibly, the inlet to the cavity has a plurality of injectors spaced around the periphery of the cavity. Possibly, the injectors are directed towards a centre of the cavity or are parallel to each other. Possibly, the injectors are of different sizes. Generally, the inlet is configured to provide a substantively laminar fluid flow across the flow cell from one side to the other. Typically, the fluid is a liquor or media for cultured cells.

There now follows by way of example only a detailed description of the present invention with reference to the accompanying drawings in which:

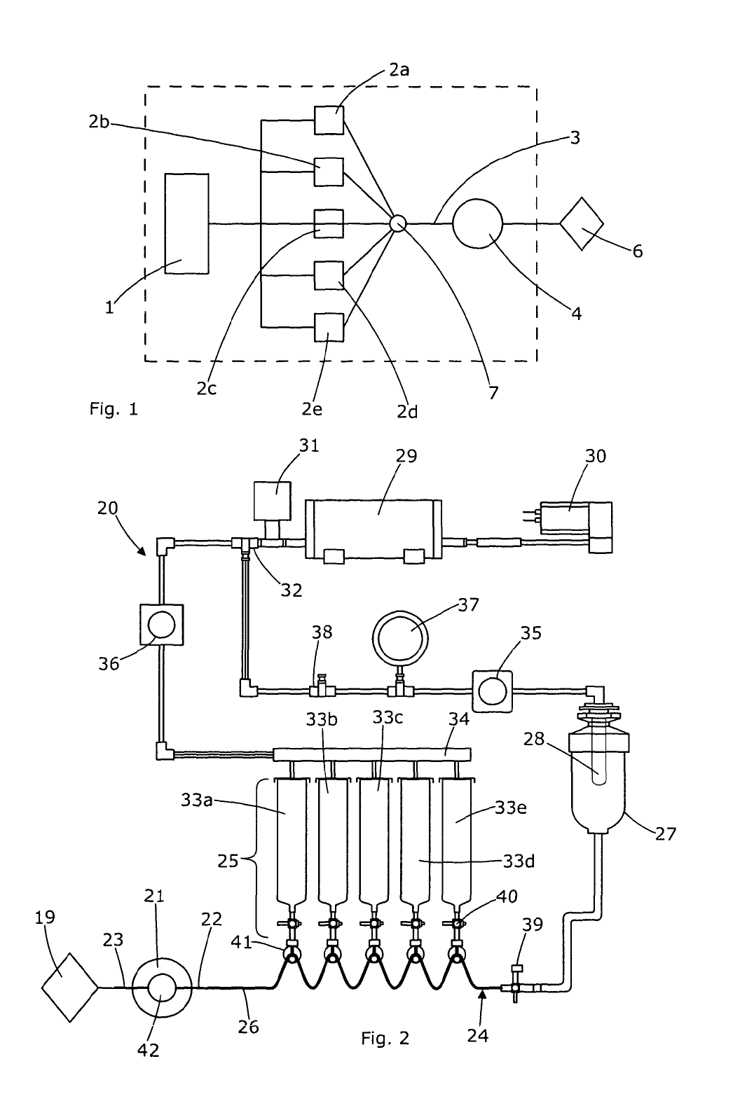

FIG. 1 is a schematic illustration of a perfusion system in which an observation cell is illustrated utilised in an observation cell arrangement in accordance with aspects of the present invention;

FIG. 2 is a perspective view of an observation cell arrangement in accordance with aspects of the present invention;

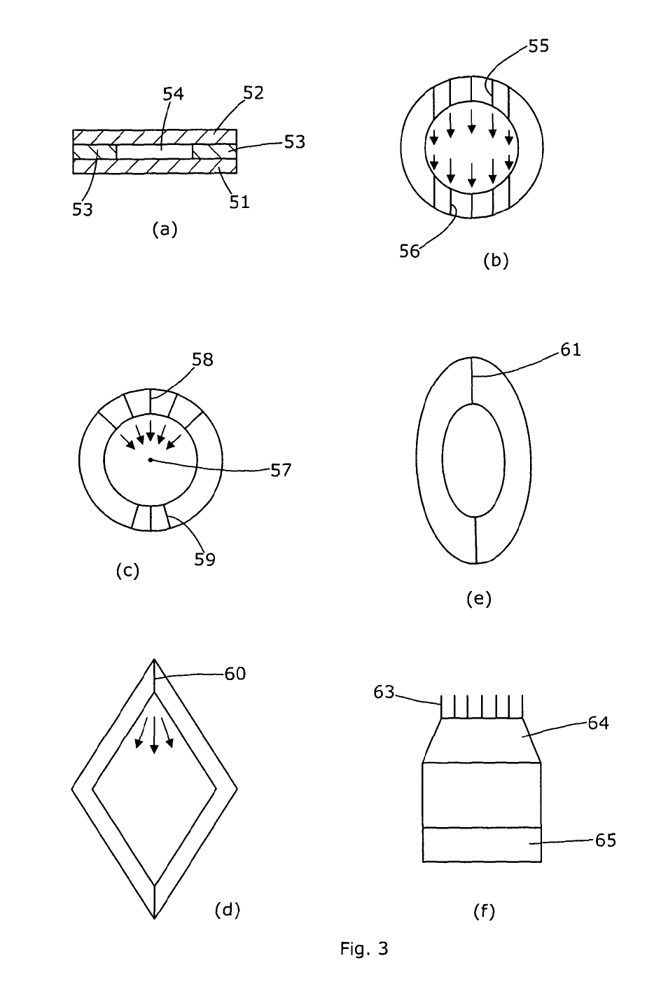

FIG. 3 provides schematic illustrations of alternate observation flow cells in accordance with aspects of the present invention;

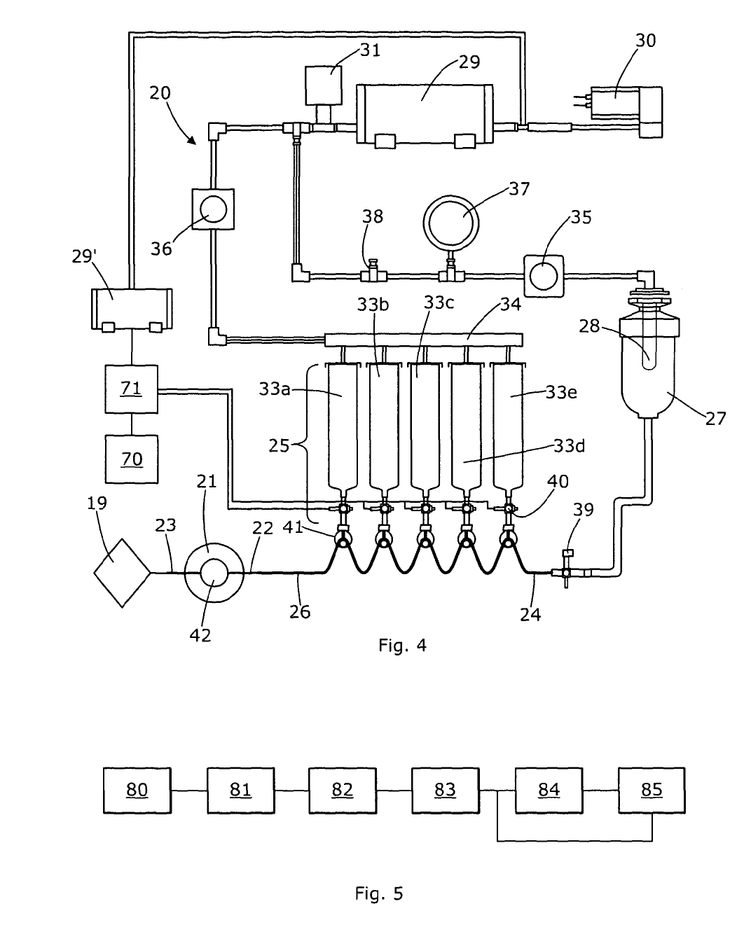

FIG. 4 shows a second embodiment of an observation cell arrangement; and

FIG. 5 shows a flow chart illustrating an exemplary method of operating the arrangement.

As indicated above making reliable observations in a flow perfusion system is difficult due to perturbations in the fluid flow. It is particularly problematic in arrangements that include confocal microscopic observation, as the potential perturbations create great difficulties due to the fine focus of such confocal microscopic systems. It will be understood that the focal plane for such confocal microscopic systems may be limited to 1 μm (or less) such that pressure pulsing and other changes will distort the image temporarily or for a period of time which may obscure observations necessary for proper analysis of cultured cell systems. Ideally a consistent laminar flow through a cultured cell system would be provided. Also, in arrangements that use fluorescence detectors or other measurement equipment, perturbations in the flow can render the measurements/observations inaccurate as the sample of interest can be moved out of view or focus or obliterated completely.

Previous systems which depend upon peristaltic pumping inherently create pressure waves which cause image distortion particularly under confocal microscopic analysis as the changes in fluid flow rate pass through a flow cell within which observation is achieved. Such an observation window generally comprises a flow cavity or cell. Typically, two plate elements sandwiching a collar or ring with a hollow centre within which the flow cavity or chamber is defined. The cavity or chamber or cell has an inlet and an outlet through which the fluid in the form of a culture support medium passes. Problems with regard to image distortion are further exacerbated when real time observation is required. Real time observation requires an ability to substantially observe changes in the cultured cells over a period of time such that periods of distortion when an image cannot be obtained inherently reduces the accuracy of determining the real time effects upon a cultured cell system.

Aspects of the present invention aim to provide a pressure driven perfusion system which can deliver a continuous smooth laminar flow of fresh cell culture medium to sustain cultured cells within a viewed portion of a flow cell which acts as an observation chamber. It will be understood that other factors such as a constant temperature and other environmental conditions can also be maintained within the arrangement. Furthermore by specific control of the pressure regime it will be understood the desired flow rates through the flow cell can be adapted dependent upon operational requirements. Although described principally with regard to cultured cells, it will be appreciated other situations where an observation cell may be sustained or require a fluid flow may also use an arrangement in accordance with aspects of the present invention.

Ideally, and as described with regard to an embodiment of the present invention, the observation cell arrangement also includes means to provide dosing of the various substances, such as prospective drug candidates, to the fluid flow into the observation flow cell to determine their effects upon the cultured cells or otherwise within the flow cell. Such introduction of dosing in accordance with aspects of the present invention can be achieved without affecting the fluid flow rate, pressure and temperature substantially as presented within the flow cell and therefore the effects of such changes will not be relevant to the observations in addition to avoiding problems with regard to the images being distorted by such variables. By such an approach real time confocal microscopic imaging of cultured cells or otherwise within the flow cell can be achieved whilst maintaining perfusion of sustaining media and other substances to the flow cell. By such an approach real time analysis of the cultured cells within the flow cell is achieved with limited if any image distortion.

FIG. 1 provides a schematic illustration of a perfusion observation cell arrangement utilised for cultured cells. Thus, within a perfusion arrangement a pressure source 1 for generally a number of fluid media reservoirs 2 is provided. A pump, which as indicated traditionally is a peristaltic pump, in such circumstances drives fluids through an inlet 3 to an observation flow cell 4 and then through an outlet 5 to a run off or dump 6. It will be appreciated that generally one of the reservoirs 2a will provide a basic systemic fluid media flow through the cell 4 in normal operation whilst other reservoirs 2b to 2e will have different fluid contents in order that the effects of such variations in the fluid content as presented in the cell 4 can be observed. Generally a valve 7 is provided to switch between the reservoirs 2 to alter the fluid flow source to the cell 4.

It will be understood that it is utilisation of pumps such as peristaltic pumps with regard to the flow driver 1 and switching by the valve 7 which can create pressure pulse perturbations in the fluid flow as presented to the cell 4. The cell 4 itself will be subject to observation by a confocal microscope, for example, and as indicated above such microscopes will be susceptible to pressure fluctuations causing distortion of the image presented. It is avoiding such pressure variations which deviate away from the ideal laminar flow which aspects of the present invention attempt to address.

FIG. 2 provides a schematic illustration of an observation cell arrangement in accordance with aspects of the present invention. The arrangement 20 comprises a flow cell 21 with a cavity inlet 22 and a cavity outlet 23 leading to a dump 19. The cavity inlet 22 being associated with a first, systemic, flow supply path 24 and a plurality of further flow supply paths comprising dosing flow supply paths 25a-e, all associated and connected in parallel to join and form a fluid delivery line 26. The fluid delivery line 26 is connected to the inlet 22. The dump 19 is required to maintain consistency of flow and to minimise perturbations to the focal plane. The dump is therefore not a closed cell and is open to atmosphere.

The first, systemic, supply path 24 includes a fluid vessel 27. A bulk of fluid is contained within the vessel 27 and pressurisation of the fluid is provided through a diaphragm 28 associated with a pressurisation source 29. The pressurisation source 29 comprises a reservoir adapted to be charged with pressurised air by a pump 30. A pressure switch 31 is provided prior to a parallel junction 32.

The parallel junction 32 transfers the pressurized air to the diaphragm 28 in parallel to the dosing supply paths 25 constituted by vessels 33. The pressure acts on the fluid in the vessel 27 (through the diaphragm) and dosing supply paths 25 to urge fluid through the delivery line 26 and the inlet 22 to the flow cell 21.

The pressure to the dosing supply paths 25 is delivered by a common manifold 34 to the vessels 33. A flow regulator 35 is provided between the junction 32 and the first vessel 27. A further flow regulator 36 is provided between the junction 32 and the manifold 34. The flow regulators 35, 36 regulate the pressure supplied to the vessel 27 and to the further, dosing flow supply paths 33. A pressure gauge 37 is also provided in line with the flow regulator 35 to enable monitoring of the pressure supplied to the first flow supply vessel 27 and a pressure relief valve 38 provides added reliability and safety. Through operation of the valve 39 for the first, systemic supply path 24 and respective valves 40 for the respective dosing paths 25, the fluid flow through the inlet 22 from the delivery line 26 is inter-leaved to maintain a consistent desired flow rate. The respective parallel flows from the respective paths 24, 25 can sustain a substantially consistent flow rate through the cell 21 in use as the paths can be balanced.

It will be understood that simultaneous opening and closing of the valves 39, 40 will result in effectively the same flow pressure and flow rate being maintained to the inlet 22. If required, adjustment of valves 36 can also produce differing flow rates from vessels 33 to that of the main, first supply through 39. Flushing chambers 41 are provided to act as “wells” which dampen switching time mis-alignments between operation of the valves 39, 40. It will be understood within the flushing chambers 40 a volume of fluid will be maintained such that if there is a slight misalignment between operations of the valves 39, 40 the volume of liquid within the chambers 41 in such circumstances will maintain a continuous smooth flow.

The pressure in the pressure reservoir 29 is controlled by a feed back loop (not shown) between the reservoir 29 and the pump 30 and the pressure switch 31. The pressure switch 31 will initiate the pump 30 should the pressure in the reservoir 29 fall below a threshold level. Further, when there is sufficient pressure in the reservoir 29, the pressure switch turns off the pump 30. Thus, as the pressure in the reservoir is maintained, there may be no noticeable pressure change to alter flow through the flow cell. The reservoir 29 in this example has a volume of 0.51 m³ and is maintained at a pressure of 1.8 bar (180 kPa).

The vessel 27 provides a relatively massive source of fluid pressurised by the diaphragm 28 through the pressurisation source 29. The diaphragm thus forms a gas pressurised displacement member, which allows air to be used as the pressure transfer fluid without contamination. The pressurisation of the fluid within the vessel 27 is consistent throughout an operational time period and therefore the systemic pressurisation and fluid flow to the inlet 22 is consistent during that time. Such consistent pressurisation will cause a consistent fluid flow which will be laminar across an observation window 42 or at least an observation portion of that window. The vessel 27 can be arranged to hold a litre of fluid.

By balancing the pressurisation within the dosing paths 25, constituted by vessels 33 and valves 40, with the systemic pressurisation in the systemic flow path 24, it will be understood that switching of the valves 39, 40 between on and off respectively would maintain the same desirable fluid pressure along the delivery line 26 to the inlet 22. Generally the pressure for fluid flow to the inlet 22 will be provided to fluid in the vessel 27. Dosing into that flow will be the further dosing paths 25 for short periods of time. In such circumstances an aliquot of fluids in the flow through the inlet 22 will be taken from the respective dosing paths 25 and driven on by return of systemic flow pressure through the valve 39 with fluid from the vessel 27 in use. When that aliquot of dosed drug or other substance enters the flow cell 21 it will act upon the cell culture within that cell and in particular as viewed through window 42 to enable through a confocal microscope, or other detector, the effects of a dosed drug on the cells to be determined. Further, when the systemic path 24 is decoupled by closing the valve 39 pressurisation of flow is maintained by the dosing supply path 25 as the drug enters the path.

It will be understood that knowledge of the distance between the respective flushing chambers 41 and the inlet 22, along with size and fluid flow rates, will enable determination of the time of dosing into the flow for the supply 26 to determine when that aliquot of drug or other substance enters the cell 21. In such circumstances it will also be understood that the flushing chambers 41 will act to wash and flush the drug or other substance dosed through the path 25 into the fluid presented to the cell culture within the cell 21. To flush the drug through the arrangement, only fluid from the first fluid path 24 is passed through to the fluid delivery line 26. The fluid from the vessel 27 will enter each and every flushing chamber 41 thereby flushing any drugs through. The arrangement of the invention is particularly advantageous as the speed of dilution in the chamber is high.

It will be understood that the actual flow rate through the inlet 22 will be determined by the common pressurisation source 29 and regulators 35,36. In such circumstances by increasing the pressurisation within the source 29 greater flow rates may be achieved. There will be balance between the pressurisation created by expansion of the diaphragm 28 within the vessel 27 and pressurisation to the dosing paths 25 through the common manifold 34. Balance will occur between the respective pressurisation paths to the supply paths 24, 25. In such circumstances should there by a slight delay between closure of valve 39 and premature opening of valve 40 as the pressurisation in the respective paths 25, 24 is substantially balanced there will be neither fluid flow into the other path nor out of the path due to pressure disparities. In such circumstances injection of the aliquot of drug of other substance into the flow to the cell 21 will be precise. It will be appreciated that it is possible to open one or several or all of the valves 40 to create a mixture of flow from the plurality of vessels 33a-e.

By aspects of the present invention essentially the fluid flow across the cell 21 will be substantially consistent. An objective will be to attempt to provide a steady laminar flow across the observation window 42 of the cell 21 such that there are no pulses or perturbations in the flow which will distort the image. As indicated above confocal microscopes have a very thin focal plane and in such circumstances such perturbations and therefore disturbance of cultured cells will result in out of focus images unacceptable for real time observation of the effects of drugs or substances on the cells. Inherently no arrangement can be idealised and in such circumstances switching of the valves 39, 40 as well as potential differences in temperature and such factors as vibration as a result of operating the valves 39, 40 may result in some perturbations in the cell 21. Nevertheless, such perturbations will be weak, extremely short lived and of relatively minimal effect in comparison with prior arrangements.

The whole arrangement can be located within an environmental cabinet to maintain a consistent temperature.

By provision of an essentially balanced relationship between the systemic supply path and the dosing supply paths, and the reduction of pressure pulses, various tests (discussed in more detail below) can be performed with the apparatus that were not consistently possible with prior art arrangements.

In a modification, the observation cell arrangement may simply comprise the systemic flow path. In such circumstances the vessel 27 and the means of pressurisation, typically through a diaphragm 28 will be such that a relatively massive pressurisation of the system is achieved and in such circumstances consistency over the whole deployment of fluid through the systemic supply path to the cell 21 can be achieved without variations in pressure and therefore flow rates. Essentially the cultured cells in the cell cavity 21 in such circumstances can be observed for a period of time with nutrients provided by the flow media without any drug or other substance intervention. The arrangement in such circumstances purely depends upon the regulation provided by the regulator 35, with a valve 39 and the sizing of the inlet 22. Nevertheless, a preferred embodiment of aspects of the present invention marries the systemic supply path with the dosing supply paths in order to allow observation/measurement of the effects of dosing upon the cultured cells within the flow cell 21. In such circumstances creating balance in terms of pressurisation and therefore flow rate is a key aspect of maintaining the as presented flow rate to the cultured cells for less disturbance and therefore problems with regard to confocal microscope image distortion, for example.

A further modification includes maintaining a sustaining fluid flow to the cells by having two systemic supply paths with vessels so that when one empties the other can be switched into supply to allow the first to be refilled. A number of systemic supply paths may be provided with automatic switching for long term maintenance of a supply to the flow cell and so the cultured cells.

As illustrated in FIG. 2, the cell 21 has an inlet 22 and an outlet 23 which are substantially matched. This matching will be in terms of size such that the flow into the cell will be equalised by the flow out of the cell 21 again to maintain a steady state with laminar flow across the cell from one side to the other. As illustrated generally the cell will comprise a hollow doughnut shape with a central cavity created between two cover elements sandwiching a ring within which the inlet 22 and outlet 23 are formed. Such constructions are well known and can be configured for individual confocal microscope types.

FIG. 3 provides various illustrations of alternate flow cell configurations. FIG. 3a provides a schematic cross section of a flow cell illustrating cover elements 51, 52 sandwiching a ring 53. The ring 53 defines a cell cavity 54 between the cover elements 51, 52 within which the cultured cells are maintained with a fluid medium flow across the ring 53 between inlet and outlet (not shown). The size of the cavity 53 as well as the cell 50 will be determined by operational requirements. The creation of the cavity 54 by a cross section of a close cell construction as depicted in FIG. 3a is dependent upon mounting within an appropriate microscope.

As indicated above creation of a laminar flow across a cell is important. Generally this can be achieved as illustrated in FIG. 2 through a single inlet and a single outlet. Alternatively, as illustrated in FIG. 3b and FIG. 3c, a plurality of inlets and/or outlets can be provided. As illustrated in FIG. 3b the inlets 55 and outlets 56 may be arranged in opposed pairs in order that flow across the cell is substantially aligned in the direction of the arrowheads depicted. Alternatively as depicted in FIG. 3c inlets may be arranged to be directed towards a point 57 within the cavity from respective inlets 58 towards outlets 59 in order that again a certain flow across the cell is created for observation.

The observation window of the flow cell is typically round. Alternatively, as depicted in FIG. 3d, a flow cell may be created which has a diamond cross section such that a single inlet 60 may create a certain flow profile across the cell desirable for observation. Further, as depicted in FIG. 3e, the cell may have an oval cross section again to create a flow profile across the cell between an inlet 61 and an outlet for better observational stability.

Generally by provision of a balanced pressurisation and therefore flow rate through an observation flow cell arrangement in accordance with aspects of the present invention greater care can be taken with regard to the flow cell itself. Furthermore bespoke and idealised cell constructions can be created utilising the steady flow rate as created in accordance with arrangements in accordance with the present invention. Thus, as illustrated with regard to FIG. 3f a cell construction may be created which includes a plurality of inlets 63 which extend in a delta type zone 64 in order to diffuse the flow in an observation window 65 and therefore create less disturbance and perturbation in that flow in order to create a steady state which can be more readily viewed by a confocal microscope. Furthermore maintenance of that steady state may be achieved through an appropriate exit or output regulator zone 65 in the form of a multi path regulator type material, such as a mesh, avoiding any disturbance in the flow as a result of evacuation.

As indicated above by creating an observation cell arrangement in accordance with aspects of the present invention in which flow pressurisation is substantially balanced and steady as well as creating a flow cell which is shaped through its input and output to limit pulsation and perturbation in the flow. A steady flow across the cell is achieved and therefore the possibility of disturbance of that flow which may alter and obscure/blur observations is reduced.

Generally the number of dosing paths can be as many as required but will be limited to avoid over complexity. Typically the flow rate will be in the order of 5 millilitres per minute with the flow pressurisation less than 6 psi. The objective is to ensure that perturbation is not created in the flow rate and particularly such perturbations do not occur when dosing is provided with regard to drugs or substances presented to the cultured cells. In such circumstances the size in terms of bore sizes with regard to the flow paths will be such that there will be consistency and furthermore consideration will be made with regard to shaping in terms of T junctions and flow paths to avoid turbulence due to flow effects within the flow paths. Generally the cell 21 will be presented downstream of the dosing paths and the systemic path to allow a degree of stabilisation within the supply path 26 in any event.

Modifications and alterations to aspects of the present invention will be understood by persons skilled in the technology. As the present invention can relate to injecting drugs into a flow of systemic fluid it will be understood that the dosing supply paths will create an aliquot or slug of fluid which passes along the delivery line 26 rather than fluids being mixed from each dosing path. Normally the dosing paths will be substantially replications of each other with the contents of the vessels 33 altered rather than the paths themselves. However alternatively it will be understood that some drugs may require higher volumes of dosing or concentrations and therefore different sizes and shaped dosing paths may be created with appropriate balance achieved through regulation from the common manifold 34 and pressurisation source 29.

To be used, the arrangement 20 only requires the connection of an electricity supply. The pump 30, powered by the electricity, can then charge the reservoir 29 with air. The electricity supply also provides power to an electric heater (not shown) that maintains the arrangement at a constant temperature, such as substantially 37° C., within an environmental cabinet (not shown).

With reference to FIG. 5, the vessel 27 can then be filled (or partly filled) with systemic fluid, as represented by step 80. Pump 30 is then actuated in order to charge the reservoir 29 with pressurised air, as represented by step 81. Cultured cells, for example, can then be placed in the flow cell 21, arranged to be visible through the observation window, as represented by step 82. The valve 39 is opened to permit a flow of systemic fluid, illustrated as step 83, which typically contains nutrients to maintain the cultured cells, to perfuse through the flow cell 21. The flow is achieved due to the pressurised air from the reservoir 29 passing through flow regulator 35 and acting on the diaphragm 28 in the first vessel 27. This causes the diaphragm to bear upon the fluid contained in the first vessel 27 urging it to flow through the first flow supply path 24, in to the delivery path 26 and into the flow cell 21. It will be appreciated that the arrangement can operate over a range of flow rates, such as between 0.06 ml/min to 20 ml/min, for example.

Examination of the cells and any tests that may be required can now be performed as represented in step 84. A number of tests can be performed with the arrangement and an example of some of them will be described below. Should the pressure in the reservoir 29 drop to below a threshold level, the pressure switch 31 will actuate and start the pump 30, as represented at step 85. Any pressure pulses generated by operation of the pump 30 are substantially absorbed by the mass of air present in the reservoir 29 thereby preventing any pressure pulses affecting the flow seen at the flow cell 21. The method now returns to step 84 as the examination of the sample can be continued, uninterrupted and undisturbed by the recharging of the reservoir. The flow rate present through the flow cell can be controlled with valve 39. Thus, the user can set the flow rate to an appropriate level to maintain the cultured cells.

A protein sheer test is useful in the field of tissue engineering and is used to evaluate how strongly bonded cells are to a support. The test involves introducing a protein, mounted on a support structure, into the flow cell and introducing the systemic flow by actuation of valve 39 at a first flow rate. The flow rate from the first flow supply path can then be increased while observations/measurements are made of the protein. The increases may be in discrete steps or a continual increase in flow rate. The flow rate that causes the protein to sheer from its support can then be determined. The increase in flow rate can be achieved by actuation of the valve 39 or valve control system 70 (discussed below).

A further test is a kinetic test in which cultured cells are placed in the flow cell and observations/measurements are made of how chemicals or antibodies or proteins, for example, bind and unbind to the surface of the cells. Thus, the cells are introduced into the flow cell 21 and the systemic supply initiated by valve 39. A chemical or antibody or protein can then be introduced from one of the dosing supply paths, by actuation of the corresponding valve 40. The dosing supply path may supply the flow cell in addition to the first flow supply path. In this case, the common reservoir 29 will balance the pressure supplied to each to maintain a steady flow rate through the flow cell as it pressurises both supplies. Further, the first flow supply path may be stopped while one of the dosing supply paths is providing the flow.

Before the dosing supply paths 25 are opened by the valves 40, the valve 39 may be throttled to temporarily reduce the flow rate through the flow cell 21. Once the dosing supply path valve is opened, the flow rate may be returned to its previous level. This is useful as this small user initiated pulse or “visual trigger” can be useful in identifying when the fluid of the dosing supply path is introduced to the flow cell 21.

A further test is an allosteric test in which the binding of a chemical/molecule/antibody to a cell protein is influenced by the presence of a further substance that acts at a different site on that protein. This allows for conditions of dilution (particularly at infinite dilution or approaching infinite dilution) to be applied so that the washout of the first substance from the cells can be monitored in real time. In addition, if a second substance is applied during this washout phase, the allosteric effect of a substance (acting at a separate site on a cell membrane protein to the first substance i.e. acting at an allosteric site) on the washout of the first substance can be monitored. As the arrangement 20 includes multiple dosing supply paths, several different substances can be loaded therein so that their effects can be evaluated.

A further advantage of the arrangement is that it can be flushed of drugs introduced from the further supply lines, as discussed above. The arrangement allows the drug to be diluted down very quickly by flushing fluid through from vessel 27 until any drug present in the fluid delivery line and thus the flow cell is so dilute that the drug cannot rebind to the cell surface receptor.

It will be appreciated that the arrangement is particularly useful for performing a method of identifying drugs. The reservoir 29 and pump 30 arrangement along with the pressure regulation 35, 36 provides a smooth pressurisation of the first and dosing supply paths that allows the above tests to be performed reliably. Further, the apparatus is particularly cost effective. Candidate drugs can be placed in each of the dosing supply paths and introduced to the flow cell and the cells therein in turn or in combination.

A second embodiment is shown in FIG. 4. The same reference numerals have been used for the same parts. While in the forgoing embodiments the valves 39 and 40 are controlled manually, in this embodiment, they are controlled by a control system that uses pressurised air to open, close and adjust the valves. The control system 70 and a valve controller 71 are connected to each of the valves 40. The valve controller 71 receives pressure from a reservoir 29′ similar to 29. The pressure reservoir 29′ is charged with compressed air by the pump 30, although it may be provided with a separate pump. It is advantageous to separate the pressure source for the control system 70/valve controller 71 and the pressure source 29 to minimise the risk of pressure pulses or changes affecting the fluid flow through the cell. The valve controller 71 is able to accurately control the valves 39, 40 on instructions from the control system 70 to open, close and control flow through the cell 21. The control system 70 may be programmable to perform a series of actions, including whether to provide a visual trigger or not. The control system can be set so that the visual trigger is sufficient for an observer to notice but not too great to the extent that the cells under observation would be disturbed. Although not shown in FIG. 4, the control system 70 also controls valve 39.

1.-37. (canceled)

38. An observation cell arrangement for flow perfusion of a sample to be observed, the arrangement comprising:
a flow cell having a cavity therein to receive the sample, the cavity having a cavity inlet and a cavity outlet, the flow cell arranged to receive a flow of fluid through the cavity from the inlet to the outlet that is directed over the sample, the cavity inlet associated with a fluid delivery line, and a first flow supply path connected to the fluid delivery line via a valve, the arrangement including a pressure source to pressurise the first flow supply path, the pressure source comprising a reservoir adapted to store pressurised fluid.

39. An observation cell arrangement according to claim 38, in which the pressure reservoir receives pressure from a pump, the pressure reservoir adapted to reduce pressure pulses from the pump.

40. An observation cell arrangement according to claim 38, in which the first flow supply path includes a first fluid vessel, the fluid vessel including a diaphragm adapted to drive fluid flow when acted on by pressure received from the pressure reservoir, the first flow supply path optionally comprises a systemic supply path and the first fluid vessel optionally adapted to receive a systemic fluid.

41. An observation cell arrangement according to claim 38, in which the arrangement includes at least one further supply path connected to the fluid delivery line via a valve, each further supply path connected to the reservoir, each further supply path optionally including one or more of:
a well between its associated valve and the fluid delivery line, the well arranged to reduce pressure pulses on actuation of the valve; and
a dosing supply path for receiving a drug to be introduced in to the cavity.

42. An observation cell arrangement according to claim 38, in which the pressure reservoir comprises a reservoir of pressurized air.

43. An observation cell arrangement according to claim 38, in which the cavity outlet is connected to an open dump.

44. An observation cell arrangement according to claim 38, in which the flow cell includes an observation window adapted to receive an examination device selected from:
a confocal microscope for examining the sample contained in the cavity; and
a fluorescence detector arranged to detect light emitted by the sample contained in the cavity.

45. An observation cell arrangement according to claim 38, in which each of the supply paths includes a flow regulator element, the further supply paths being optionally supplied with pressure via a common manifold.

46. An observation cell arrangement according to claim 38, in which the flow cell comprises a ring between two cover plate elements.

47. An observation cell arrangement according to claim 38, in which the cavity inlet has a plurality of injectors spaced around the periphery of the cavity, the injectors being optionally directed towards a centre of the cavity or parallel to each other, the injectors being optionally of different sizes.

48. An observation cell arrangement as claimed in claim 38, wherein the inlet is configured to provide a substantively laminar fluid flow across the flow cell from one side to the other.

49. An observation cell arrangement as claimed in claim 38, wherein the cavity is round or diamond shaped or oval.

50. An observation cell arrangement according to claim 38, in which the arrangement is housed in an environmental cabinet to maintain the arrangement at a substantially constant temperature.

51. A method of performing observations with a flow perfusion apparatus comprising the steps of;
a) adding a systemic fluid to a first fluid vessel;
b) charging a reservoir with pressurised fluid;
c) adding a sample to be observed to a flow cell cavity;
d) initiating a flow of fluid through the flow cell cavity using the pressure reservoir;
e) examining the sample.

52. A method according to claim 51, in which step (e) comprises using a confocal microscope to observe the sample or using a florescence detector to detect fluorescence emitted by the sample.

53. A method according to claim 51, in which the method includes the step of making adjustments to the flow rate of at least the fluid in the first fluid vessel and making further examinations.

54. A method according to claim 51, in which the apparatus includes a further fluid supply path containing a dosing fluid, the method comprising the steps of:
i) introducing the dosing fluid and
ii) examining the effect of the dosing fluid on the sample,
the method optionally including the step of temporarily reducing the flow rate of the systemic fluid prior to the introduction of the dosing fluid and upon introduction of the dosing fluid, increasing the flow rate to provide a cue to an observer that the dosing fluid has been introduced.

55. A method according to claim 54 in which the flow rate is increased to a level substantially equal to that prior to the temporary reduction in flow rate.

56. A method according to claim 54, in which the dosing fluid is introduced in addition to the systemic fluid, while maintaining a substantially constant flow rate through the flow cell when the dosing fluid is introduced.

57. A method according to claim 54, in which when the dosing fluid is introduced the systemic fluid is stopped by simultaneous actuation of respective valves, thus maintaining a substantially constant flow rate through the flow cell when the dosing fluid is introduced.

58. A method according to claim 51 in which method includes one or more of:
the step of connecting the apparatus only to one external supply, namely an electricity supply, prior to examining the sample; and
the step of charging the pressure reservoir with pressurised fluid while the fluid is flowing through the flow cell.

59. A method of identifying a drug comprising the steps of:
a) placing cultured cells in a flow cell;
b) providing a systemic fluid flow at a first, substantially constant, flow rate over the cultured cells;
c) introducing a dosing fluid flow of a first candidate drug while maintaining the substantially constant first flow rate over the cells;
d) observing the effect of the drug on the cultured cells;
e) identifying the drug as being effective if the effect on the cultured cells fulfils predetermined criteria.

60. A method according to claim 59, in which the method includes the step of temporarily reducing the flow rate of the systemic fluid prior to introducing dosing fluid, and, upon introduction of the dosing fluid, increasing the flow rate to provide a cue to an observer that the dosing fluid has been introduced.

61. A method according to claim 59, in which the method includes one or more of:
performing a kinetic test;
introducing a first substance to perfuse the cells, and observing the effect on the cells, introducing a second substance to perfuse the cells in addition to the first substance and observing how the second substance influences the effect of the first substance on the cells;
introducing a first substance to perfuse the cells, diluting the first substance during a “washout” phase, and observing the effect on the cells.

62. A method according to claim 61, in which the method step additionally includes introducing a second substance, different to the first substance, during the “washout” phase and observing the resulting allosteric effect on the cells.

63. A method according to claim 59, in which the method includes the step of charging the pressure reservoir with pressurised fluid during the method of identifying a drug.
